(19) United States
(12) Patent Application Publication
(10) Pub. No.: US 2023/0222940 A1
(43) Pub. Date: Jul. 13, 2023

(54) VIRTUAL TEACHING SYSTEM FOR DENTAL PERIAPICAL X-RAY FILM, METHOD FOR ACQUIRING VIRTUAL PERIAPICAL X-RAY FILM, COMPUTER READABLE STORAGE MEDIUM AND ELECTRONIC DEVICE

(71) Applicant: Nanjing Jiuzhenwei Medical Technology Co., Ltd., Jiangsu (CN)

(72) Inventors: Weibin SUN, Jiangsu (CN); Zhuoyi LIAO, Jiangsu (CN); Xun CAO, Jiangsu (CN); Yidi ZHANG, Jiangsu (CN)

(73) Assignee: Nanjing Jiuzhenwei Medical Technology Co., Ltd., Jiangsu (CN)

(21) Appl. No.: 17/718,335

(22) Filed: Apr. 12, 2022

(30) Foreign Application Priority Data
Jan. 12, 2022 (CN) 202210031048.3

(51) Int. Cl. G09B 23/28; A61B 6/00; A61B 6/14

(52) U.S. Cl. CPC G09B 23/283; A61B 6/54; A61B 6/4458; A61B 6/5205; A61B 6/14

(57) ABSTRACT

A virtual teaching system for a dental periapical X-ray film and a virtual periapical X-ray film acquisition method is provided. The system includes a workstation and a dental radiography machine. The dental radiography machine includes a mounting plate, a stand column, a head fixing device, a five-axis robotic arm, a bulb tube, and a seat; the stand column is disposed on the top side of the mounting plate, and the head fixing device and the seat are respectively fixed on the upper and lower portions of the stand column; and the five-axis robotic arm includes five joint modules and five connecting rods, and the two ends of the five-axis robotic arm are respectively connected to the top portion of the stand column and the bulb tube. The workstation is communicatively connected to the dental radiography machine.

### CROSS-REFERENCE TO RELATED APPLICATION

This application claims the priority benefit of China application serial no. 202210031048.3, filed on Jan. 12, 2022. The entirety of the above-mentioned patent application is hereby incorporated by reference herein and made a part of this specification.

### TECHNICAL FIELD

The present application relates to the technical field of dental periapical X-ray films, and in particular to a virtual teaching system for a dental periapical X-ray film and a method for acquiring a virtual periapical X-ray film.

### BACKGROUND

A periapical X-ray film is an X-ray film taken by a dental radiography machine, which can effectively help dentists to diagnose dental diseases; and diseases including tooth decay, periapical periodontitis, periodontitis, etc. have corresponding imaging manifestations on the periapical X-ray film. At present, the periapical X-ray film projection method used in most hospitals and clinics of the world is the angle-bisecting technique. The principle of the technique is that: because the oral cavity is a small space that cannot be viewed directly, it is usually difficult to determine the angle of each tooth in a patient's mouth, and it is difficult to make a film parallel to the teeth, but an angle is formed therebetween. When the centerline of an X-ray bulb tube is perpendicular to the angular bisector of this angle, an isosceles triangle is formed, and then the actual length of the tooth is equal to the projection length of the tooth on the film, which is the principle of the angle-bisecting technique.

The periapical X-ray film is the most commonly used image-aided diagnosis means in clinic, and every dentist must master the angle-bisecting technique. Therefore, the teaching and assessment of periapical radiography is very important. However, X rays are radioactive to human health, and the angle-bisecting technique requires accumulation of a lot of experience. Therefore, for a long time, the learning and assessment of the angle-bisecting projection technique is a challenge. In the conventional methods, medical students can only mechanically memorize the experience angle and cannot practice a lot due to the limitation of the X rays, and there is no direct image feedback, resulting in low learning efficiency.

For the problem of the low learning efficiency because the medical students cannot practice a lot due to the limitation of the X rays in a periapical X-ray film learning process and there is no direct image feedback in the prior art, no effective solution has been proposed in the prior art.

### SUMMARY

Embodiments of the present application provide a virtual teaching system for a dental periapical X-ray film and a virtual periapical X-ray film acquisition method, so as to at least solve the problem of the low learning efficiency because the medical students cannot practice a lot due to the limitation of the X rays in a periapical X-ray film learning process and there is no direct image feedback in the prior art.

In an embodiment of the present application, a virtual teaching system for a dental periapical X-ray film is provided, which includes a workstation and a dental radiography machine. The dental radiography machine includes a mounting plate, a stand column, a head fixing device, a five-axis robotic arm, a bulb tube, and a seat, where the stand column is disposed on a top side of the mounting plate, the head fixing device is fixed on an upper portion of the stand column, and the seat is fixed on a lower portion of the stand column; and the five-axis robotic arm includes five joint modules and five connecting rods, and one end of the five-axis robotic arm is connected to a top portion of the stand column and another end of the five-axis robotic arm is connected to the bulb tube. The workstation is communicatively connected to the dental radiography machine, so that the dental radiography machine positions standard teeth in a dentition model via the bulb tube, generates virtual projection image data of the standard teeth on a virtual plane, and sends the virtual projection image data to the workstation for rendering, to obtain a virtual periapical X-ray film corresponding to the dentition model.

In an embodiment, the five-axis robotic arm includes: a first joint module, disposed between the stand column and a first connecting rod; a second joint module, disposed between the first connecting rod and a second connecting rod; a third joint module, disposed between the second connecting rod and a third connecting rod; a fourth joint module, disposed between the third connecting rod and a fourth connecting rod; and a fifth joint module, disposed between the fourth connecting rod and a fifth connecting rod, the fifth connecting rod being connected to the bulb tube; where each of the joint modules is internally disposed with a multi-turn absolute encoder configured to acquire a number of turns of rotation of the corresponding joint module.

In an embodiment, the system further includes: a programmable logic controller (PLC) and a switch that are successively disposed between the dental radiography machine and the workstation, where the number of turns of rotation of each of the joint modules is sent to the switch by means of the PLC and then sent to the workstation via the switch; and the workstation determines coordinates of the dentition model in a bulb tube coordinate system and a rotation matrix between a world coordinate system and the bulb tube coordinate system according to the number of turns of rotation of each of the joint modules, a length of each of the joint modules, a length of each of the connecting rods, and coordinates of the dentition model in the world coordinate system, and then performs rendering to obtain the corresponding virtual periapical X-ray film.

In an embodiment, the dental radiography machine further includes: casters, mounted on the bottom side of the mounting plate.

In another embodiment of the present application, a method for acquiring a virtual periapical X-ray film is further provided, which includes: acquiring initial rendered images of a dentition model at different angles; taking an image of the dentition model at a preset position by using a bulb tube of a dental radiography machine, where the dental radiography machine includes a five-axis robotic arm, the five-axis robotic arm includes five joint modules and five connecting rods, and an end portion of the five-axis robotic arm is connected to the bulb tube; determining coordinates of the dentition model in a bulb tube coordinate system and a rotation matrix between a world coordinate system and the bulb tube coordinate system according to a number of turns of rotation of each of the joint modules, a length of each of the joint modules, a length of each of the connecting rods, and coordinates of the dentition model in the world coordinate system; and obtaining a virtual periapical X-ray film corresponding to the dentition model according to the coordinates of the dentition model in the bulb tube coordinate system, the rotation matrix between the world coordinate system and the bulb tube coordinate system, and a spatial posture and the Euler angle of the bulb tube, and with reference to the initial rendered images of the dentition model.

In an embodiment, the step of determining the coordinates of the dentition model in the bulb tube coordinate system and the rotation matrix between the world coordinate system and the bulb tube coordinate system according to the number of turns of rotation of each of the joint modules, the length of each of the joint modules, the length of each of the connecting rods, and the coordinates of the dentition model in the world coordinate system includes: determining an angle of rotation of each of the joint modules according to the number of turns of rotation of each of the joint modules; and determining the coordinates of the dentition model in the bulb tube coordinate system and the rotation matrix between the world coordinate system and the bulb tube coordinate system according to a DH coordinate system of the robotic arm and a transformation matrix.

In an embodiment, after the virtual periapical X-ray film corresponding to the dentition model is obtained, the method further includes: determining whether there is a deviation between a real X-ray film and the virtual periapical X-ray film, where the real X-ray film is an X-ray film of the dentition model taken by an X-ray machine, and when the X-ray machine takes the real X-ray film, a placement position of the dentition model and an angle of the bulb tube are consistent with those when the dental radiography machine takes the virtual periapical X-ray film; and if it is determined that there is a deviation, adjusting the placement position of the dentition model and the angle of the bulb tube according to the deviation between the real X-ray film and the virtual periapical X-ray film.

In an embodiment, the step of determining whether there is a deviation between the real X-ray film and the virtual periapical X-ray film includes: keeping an angle of rotation of each joint module of the X-ray machine consistent with an angle of rotation of each of the joint modules of the dental radiography machine; operating the X-ray machine and the dental radiography machine simultaneously, to obtain the real X-ray film and the virtual periapical X-ray film; comparing the real X-ray film with the virtual periapical X-ray film in terms of a root length and an overall contour offset degree; and if a difference in the root length is greater than 1 mm or a difference in the overall contour offset degree is greater than or equal to 0.2, determining that there is a deviation between the real X-ray film and the virtual periapical X-ray film.

In an embodiment of the present application, a computer readable storage medium is further provided, the storage medium storing a computer program, where the computer program is configured to perform steps of any of the foregoing method embodiments during running.

In an embodiment of the present application, an electronic device is further provided, which includes a memory and a processor, the memory storing a computer program, where the processor is configured to perform steps of any of the foregoing method embodiments by running the computer program.

Embodiments of the present application provide a virtual teaching system for a dental periapical X-ray film, which includes a workstation and a dental radiography machine. The dental radiography machine includes a mounting plate, a stand column, a head fixing device, a five-axis robotic arm, a bulb tube, and a seat, where the stand column is disposed on the top side of the mounting plate, and the head fixing device and the seat are respectively fixed on the upper and lower portions of the stand column; the five-axis robotic arm includes five joint modules and five connecting rods, and the two ends of the five-axis robotic arm are respectively connected to the top portion of the stand column and the bulb tube. The workstation is communicatively connected to the dental radiography machine, so that the dental radiography machine positions standard teeth in a dentition model via the bulb tube, generates virtual projection image data of the standard teeth on a virtual plane, and sends the virtual projection image data to the workstation for rendering, to obtain a virtual periapical X-ray film corresponding to the dentition model. The present application solves problems such as X-ray radiation, no direct image feedback, and low learning efficiency in the conventional teaching of angle-bisecting projection of the periapical X-ray film. By the arrangement of the five-axis robotic arm (having multiple connecting rods and joints), the virtual teaching system for a dental periapical X-ray film provided by the embodiments of the present application can produce, by rendering in real time, a high-accuracy virtual periapical X-ray film with a precision up to one thousandth of a degree when the student operates the dental radiography machine-based teaching platform, thus greatly improving the learning efficiency of the student when learning the angle-bisecting projection technique and avoiding the harm from the X rays.

### BRIEF DESCRIPTION OF THE DRAWINGS

The accompanying drawings described herein are used to provide a further understanding of the present application and constitute a part of the present application. The illustrative embodiments in the present application and the related descriptions are used to explain the present application, and do not constitute improper limitations for the present application. In the accompanying drawings:

FIG. 1 is an overall schematic structural diagram of an optional virtual teaching system for a dental periapical X-ray film according to an embodiment of the present application;

FIG. 2 is a schematic structural diagram of an optional dental radiography machine according to an embodiment of the present application;

FIG. 3 is a schematic structural diagram of an optional five-axis robotic arm according to an embodiment of the present application;

FIG. 4 is a schematic diagram of an internal structure of an optional first joint module according to an embodiment of the present application;

FIG. 5 is a schematic diagram of an optional robotic arm coordinate system according to an embodiment of the present application;

FIG. 6 is a flowchart of an optional method for acquiring a virtual periapical X-ray film according to an embodiment of the present application; and

FIG. 7 is a schematic structural diagram of an optional electronic device according to an embodiment of the present application.

### DETAILED DESCRIPTION OF THE EMBODIMENTS

The present application will be described below with reference to the accompanying drawings and specific embodiments. It should be noted that, the embodiments in the present application and the features in the embodiments may be combined with each other without conflicts.

It should be noted that the following detailed descriptions are illustrative and are intended to further clarify the present application. Unless otherwise specified, all technical and scientific terms used herein have the same meaning as commonly understood by those of ordinary skill in the art to which the present application belongs.

It should be noted that the terms used herein are intended to describe specific implementations only and are not intended to limit the exemplary implementations according to the present application. As used herein, unless the context clearly indicates otherwise, the singular form is also intended to include the plural form. In addition, it should be also understood that, when the terms “comprising” and/or “including” are used in the present specification, they indicate the presence of features, steps, operations, devices, components, and/or combinations thereof.

It should be noted that, the terms “first”, “second”, and so on in the specification, claims and the foregoing drawings of the present application are used to distinguish similar objects, but not necessarily to describe a particular order or sequence. It should be understood that data used as such can be interchanged under appropriate circumstances, so that the embodiments of the present application described herein could be implemented in an order other than the order illustrated or described herein. In addition, the terms “comprise/include” and “have” as well as any variations thereof are intended to cover non-exclusive inclusion. For example, a process, method, system, product or device including a series of steps or units are not necessarily limited to the steps or units clearly listed, but may include other steps or units not clearly listed in or inherent to the process, method, system, product or device.

For ease of description, spatially relative terms such as “above”, “over”, “on the top of”, “upper”, etc. may be used herein to describe a spatial positional relationship between one device or feature and another device or feature shown in the drawings. It should be understood that the spatially relative terms aim to contain different orientations of the device in use or operation other than those depicted in the drawings. For example, if the device in the drawings is inverted, the device described as “over another device or structure” or “above another device or structure” will then be positioned as “under another device or structure” or “below another device or structure”. Therefore, the exemplary term “above” may include two orientations: “above” and “below”. The device can also be oriented in other ways (through 90-degree rotation or in other directions), and the spatially relative descriptions used herein are explained accordingly.

To make the objectives, technical solutions, and advantages of the present application clearer, the technical solutions in the embodiments of the present invention are clearly and completely described below with reference to the accompanying drawings in the embodiments of the present application. Apparently, the described embodiments are some rather than all of the embodiments of the present application. Based on the embodiments of the present application, other embodiments obtained by those of ordinary skill in the art without creative effort all belong to the protection scope of the present application.

In addition, it should be further noted that, for the convenience of description, the accompanying drawings only show some but not all structures related to the present application. Throughout the specification, the same or similar reference numbers represent the same or similar structures, elements or processes. It should be noted that, the embodiments in the present application and the features in the embodiments may be combined with each other without conflicts.

The terms “comprise/include” used in the present application and any derivatives thereof are intended to cover non-exclusive inclusion.

Unless clearly defined, when an element is referred to as being “connected to” another element in the present application, it can be directly connected/fixed to another element, or there may be an intervening element.

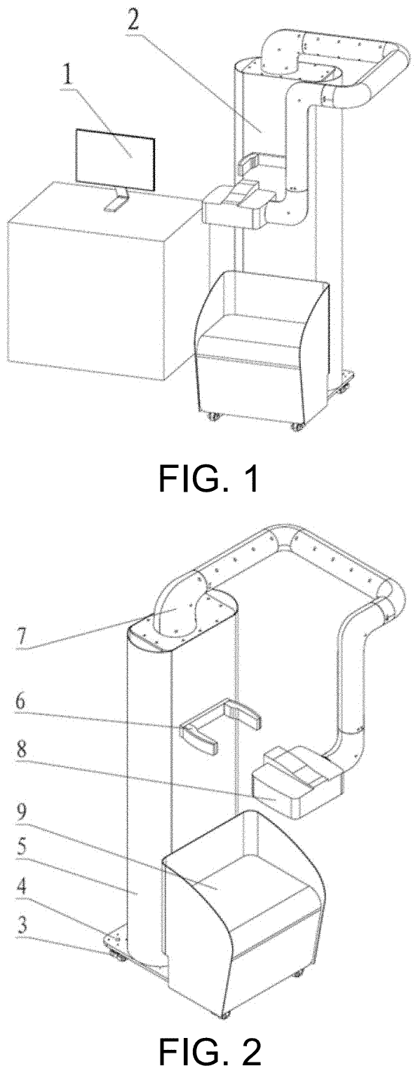

As shown in FIGS. 1 and 2, an embodiment of the present application provides a virtual teaching system for a dental periapical X-ray film, which includes: a workstation 1 and a dental radiography machine 2. The dental radiography machine includes a mounting plate 4, a stand column 5, a head fixing device 6, a five-axis robotic arm 7, a bulb tube 8, and a seat 9. The stand column 5 is disposed on the top side of the mounting plate 4, the head fixing device 6 is fixed on the upper portion of the stand column, and the seat 9 is fixed on the lower portion of the stand column 5. The five-axis robotic arm 7 includes five joint modules and five connecting rods, and one end of the five-axis robotic arm 7 is connected to the top portion of the stand column 5 and another end of the five-axis robotic arm 7 is connected to the bulb tube 8. The workstation 1 is communicatively connected to the dental radiography machine 2, so that the dental radiography machine 2 positions standard teeth in a dentition model via the bulb tube 8, generates virtual projection image data of the standard teeth on a virtual plane, and sends the virtual projection image data to the workstation 1 for rendering, to obtain a virtual periapical X-ray film corresponding to the dentition model. For ease of movement, casters may be mounted on the bottom side of the mounting plate 4. The dentition model may be a 3D model made in advance, and the standard teeth may be construed as meaning that the tooth morphology in the dentition model meet general standard requirements.

The head fixing device 6 and the seat 9 are used for fixing a teaching head model for ease of photographing. The virtual teaching system for a dental periapical X-ray film in the embodiment of the present application uses the dentition model for photographing, without involving photographing of real people.

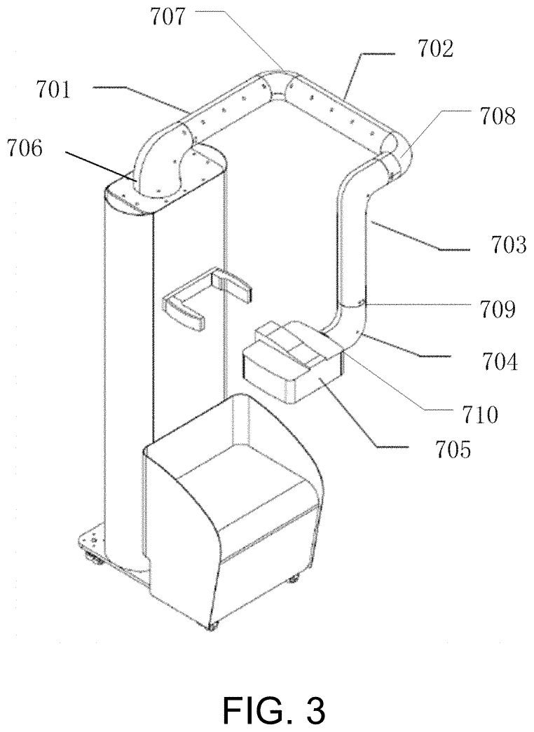

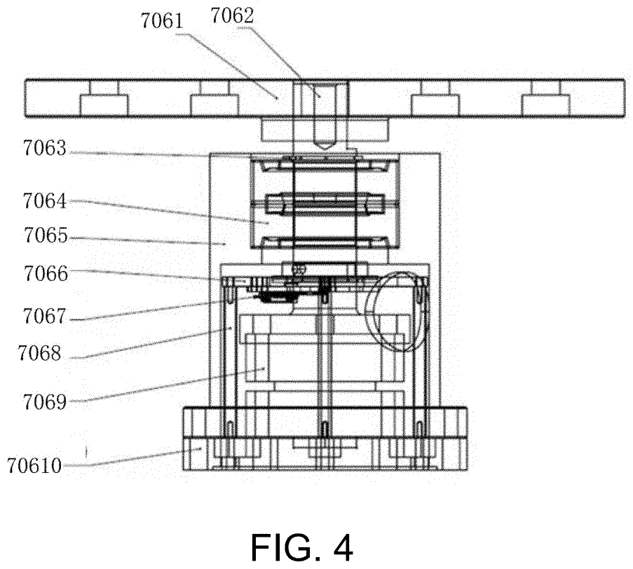

As shown in FIGS. 3, 4, and 5, in an embodiment, the five-axis robotic arm includes: a first joint module 706 (a joint 1), disposed between the stand column 5 and a first connecting rod 701 (a connecting rod 1); a second joint module 707 (a joint 2), disposed between the first connecting rod 701 and a second connecting rod 702 (a connecting rod 2); a third joint module 708 (a joint 3), disposed between the second connecting rod 702 and a third connecting rod 703 (a connecting rod 3); a fourth joint module 709 (a joint 4), disposed between the third connecting rod 703 and a fourth connecting rod 704 (a connecting rod 4); and a fifth joint module 710 (a joint 5), disposed between the fourth connecting rod 704 and a fifth connecting rod 705, the fifth connecting rod 705 (a connecting rod 5) being connected to the bulb tube. Each of the joint modules is internally disposed with a multi-turn absolute encoder configured to acquire the number of turns of rotation of the corresponding joint module.

In an embodiment, the system further includes: a PLC and a switch that are successively disposed between the dental radiography machine 2 and the workstation 1. The number of turns of rotation of each of the joint modules is sent to the switch by means of the PLC and then sent to the workstation 1 via the switch. Then, the workstation 1 determines coordinates of the dentition model in a bulb tube coordinate system and a rotation matrix between a world coordinate system and the bulb tube coordinate system according to the number of turns of rotation of each of the joint modules, the length of each of the joint modules, the length of each of the connecting rods, and coordinates of the dentition model in the world coordinate system; and then performs rendering to obtain the corresponding virtual periapical X-ray film.

By using the first joint module 706 as an example, the joint module in the present application includes: a ring plate 7061 connected to the next-level connecting rod; a transmission shaft 7062, a snap ring 7063, a bearing 7064, a fixing housing 7065, a first connecting plate 7066, a multi-turn absolute encoder 7067, a fixed rod 7068, a damper shaft 7069, and a second connecting plate 70610. The multi-turn absolute encoder, as a core part, has a precision of up to 19 bits and can acquire an angle of rotation of each joint module by calculating the number of turns of rotation, thus facilitating conversion between different robotic arm coordinate systems. In this way, the deficiency that the existing infrared pulse system is easily blocked and cannot be positioned is avoided, and further the precision of the angle can be greatly improved, where the precision of less than one thousandth of a degree can be easily achieved.

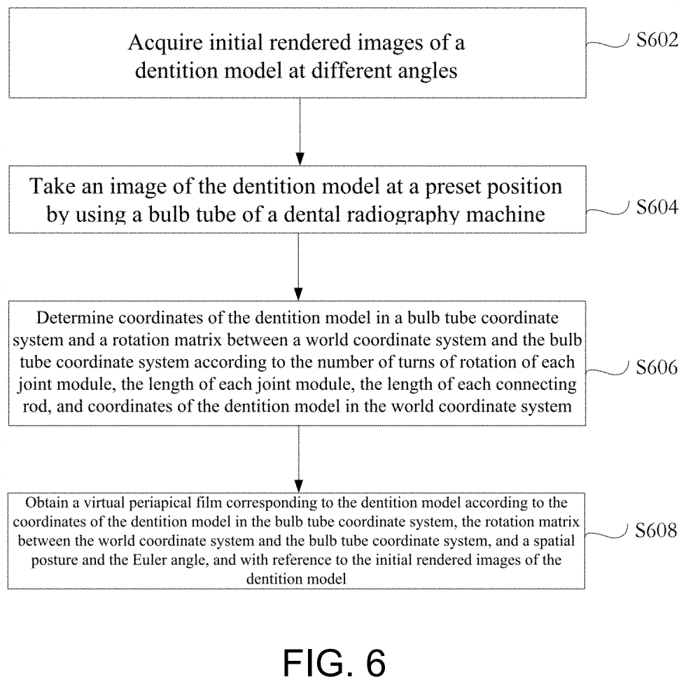

In another embodiment of the present application, a method for acquiring a virtual periapical X-ray film is further provided, which, as shown in FIG. 6, includes the following steps.

Step S602: Initial rendered images of a dentition model at different angles are acquired.

Step S604: An image of the dentition model at a preset position is taken by using a bulb tube of a dental radiography machine, where the dental radiography machine includes a five-axis robotic arm, the five-axis robotic arm includes five joint modules and five connecting rods, and an end portion of the five-axis robotic arm is connected to the bulb tube.

Step S606: Coordinates of the dentition model in a bulb tube coordinate system and a rotation matrix between a world coordinate system and the bulb tube coordinate system are determined according to the number of turns of rotation of each of the joint modules, the length of each of the joint modules, the length of each of the connecting rods, and coordinates of the dentition model in the world coordinate system.

Step S608: A virtual periapical X-ray film corresponding to the dentition model is obtained according to the coordinates of the dentition model in the bulb tube coordinate system, the rotation matrix between the world coordinate system and the bulb tube coordinate system, and a spatial posture and the Euler angle of the bulb tube, and with reference to the initial rendered images of the dentition model, where the Euler angle is a triad of independent angle parameters used to uniquely determine the position of a rigid body rotating at a fixed point; and briefly, refers to angles of rotation of an object about the three coordinate axes (the x, y, and z axes) of a coordinate system.

In an embodiment, after the virtual periapical X-ray film corresponding to the dentition model is obtained, the method further includes: determining whether there is a deviation between a real X-ray film and the virtual periapical X-ray film, where the real X-ray film is an X-ray film of the dentition model taken by an X-ray machine, and when the X-ray machine takes the real X-ray film, a placement position of the dentition model and an angle of the bulb tube are consistent with those when the dental radiography machine takes the virtual periapical X-ray film; and if it is determined that there is a deviation, adjusting the placement position of the dentition model and the angle of the bulb tube according to the deviation between the real X-ray film and the virtual periapical X-ray film. The placement position of the dentition model may be construed as a body surface mark point aligned with a laser pointer or an X-ray centerline, and the angle of the bulb tube may be construed as angles of the bulb tube about the xyz axes in a spatial coordinate system. It is required to control the variables during comparison between the virtual periapical X-ray film and the real periapical X-ray film. A laser pointer is bound to the bulb tube of the virtual machine to simulate an x-ray centerline, and a projection position of the x-ray centerline on the teeth needs to be determined when the periapical X-ray film is taken.

In an embodiment, the step of determining whether there is a deviation between the real X-ray film and the virtual periapical X-ray film includes: keeping an angle of rotation of each joint module of the X-ray machine consistent with an angle of rotation of each of the joint modules of the dental radiography machine; operating the X-ray machine and the dental radiography machine simultaneously, to obtain the real X-ray film and the virtual periapical X-ray film; comparing the real X-ray film with the virtual periapical X-ray film in terms of the root length and the overall contour offset degree; and if a difference in the root length is greater than 1 mm or a difference in the overall contour offset degree is greater than or equal to 0.2, determining that there is a deviation between the real X-ray film and the virtual periapical X-ray film.

Specifically, the forgoing method may be implemented by means of the following steps.

Step 1. Establishment of a Three-Dimensional Model of a Tooth Projection Image on a Virtual Plane

In oral and maxillofacial imaging, features of an X-ray image can be processed as “texture”. In this way, we can treat a section of a three-dimensional model as a two-dimensional image added with the “X-ray texture”, which is the core idea of our simulation of the periapical X-ray film. Specific steps are as follows:

1) Fabrication of a dentition model by filling: Resin teeth with pulp cavities are placed in corresponding positions (17-27, 37-47) in a female dentition die, super hard gypsum powder and water are mixed and added to the female die, shaking is performed to expel the excess air bubbles, and then the female die is put aside for 25 min to 30 min, to obtain a pair of upper and lower dentition models in an arrangement according to the standard model.

2) Scanning with CBCT: The upper and lower dentition models are scanned by using a CBCT machine (NewTom VGI evo), to export corresponding DICOM files, where CBCT is short for Cone beam CT, namely, cone-beam CT. Just as its name implies, the CBCT machine is cone-beam computed tomography imaging equipment, and its principle is that an X-ray generator makes a circular DR (digital projection) around a projection body with a low amount of radiation (usually the bulb tube current is around 10 mA). Then, data obtained in an “intersection” generated after multiple digital projections (180 to 360 times, varying between different products) around the projection body is reconstructed in the computer, to finally obtain a three-dimensional image.

3) Three-dimensional reconstruction: Three-dimensional reconstruction is performed for the obtained DCM files by using Mimics Medical 21.0, to remove air bubbles from the plaster model and extract the dentition (including the pulp cavities) therefrom. After extraction, the dentition model is stored in an STL format; and processing such as shell separation is performed by using Meshmixer software, to obtain a dentition model containing the pulp cavities (3D printing).

4) Image pre-rendering: In blender software, the tooth model is placed in a certain manner; and the camera is oriented in a direction of ray incidence and is set to orthogonal projection (namely, has no near-large and far-small effect). With reference to an actual image of a periapical X-ray film, texture is manually drawn for the model, and the material transparency of the model is set. The materials of the pulp cavity and the outer shell reflect different colors of light, where the pulp cavity part reflects dark colors, while the outer shell reflects white. The rendering process is controlled by using the python script and angular transformation is performed. The ratio by which the tooth model needs to be scaled is calculated according to the angle values and the foregoing schematic diagrams, and then rendering is performed. Pictures at various angles are rendered and then stored locally. By writing exe program, a corresponding picture is found according to the input angle and is displayed, and the pictures are linked to form a video.

Step 2. Establishment of a DH Spatial Coordinate System of the Robotic Arm

The novel periapical X-ray film virtual platform of the dental radiography machine after assembly can be regarded as a robotic arm (five-axis robotic arm) system RRRRR having five joints, which is specifically shown in the figures. The dentition model is fixed at a certain point in the world coordinate system and does not move, and the coordinates M0 of the dentition model are known in the world coordinate system. By conversion between different connecting rod coordinate systems, the coordinates of M0 in the bulb tube coordinate system (namely, the coordinate system of the connecting rod 5) and the rotation matrix between the world coordinate system and the bulb tube coordinate system can be finally calculated. By means of the rotation matrix, the workstation of the virtual teaching platform of the dental radiography machine can calculate the coordinates of the dentition model in the bulb tube coordinate system in real time, and the corresponding virtual periapical X-ray film can be obtained by rendering according to the spatial posture and the Euler angle and others.

Step 3. Detailed Explanation of Joint Modules of the Virtual Teaching Platform of the Dental Radiography Machine

For the precision of the coordinate calculation, the joints of the virtual teaching platform of the dental radiography machine must have the following characteristics: stability, precision, and non-resilience. To achieve the foregoing objectives, each joint module has several parts: a ring plate (connected to the next-level arm), a bearing snap ring, a bearing, a transmission shaft, a multi-turn absolute encoder, a damper shaft, and a fixing housing. The multi-turn absolute encoder, as a core part of the novel virtual teaching platform of the dental radiography machine, achieves a precision up to 19 bits and can acquire an angle of rotation of each joint by calculating the number of turns of rotation, thus facilitating conversion between different robotic arm coordinate systems. In this way, the deficiency that the previous-generation infrared pulse system is easily blocked and cannot be positioned is avoided, and further the precision of the angle can be greatly improved, where a precision of less than one thousandth of a degree can be easily achieved.

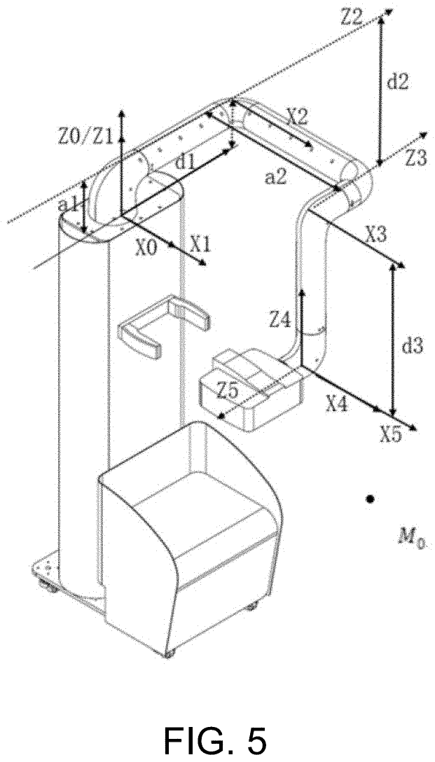

FIG. 5 is a schematic diagram of an optional robotic arm coordinate system according to an embodiment of the present application, where parameters in FIG. 5 are described in table 1 below:

TABLE 1

| No. | Parameter | Description |
|---|---|---|
| 1 | x₀ | X axis of the base coordinate system |
| 2 | x₁ | X axis of the connecting rod 1 |
| 3 | x₂ | X axis of the connecting rod 2 |
| 4 | x₃ | X axis of the connecting rod 3 |
| 5 | x₄ | X axis of the connecting rod 4 |
| 6 | x₅ | X axis of the connecting rod 5 |
| 7 | z₀ | Z axis of the base coordinate system |
| 8 | z₁ | Z axis of the connecting rod 1 |
| 9 | z₂ | Z axis of the connecting rod 2 |
| 10 | z₃ | Z axis of the connecting rod 3 |
| 11 | z₄ | Z axis of the connecting rod 4 |
| 12 | z₅ | Z axis of the connecting rod 5 |
| 13 | a₄ | Length of the common perpendicular to Z axes of |
| | | the connecting rods 2 and 1 |
| 14 | a₂ | Length of the common perpendicular to Z axes of |
| | | the connecting rods 3 and 2 |
| 15 | d₁ | Length of the common perpendicular to X axes of |
| | | the connecting rods 2 and 1 |
| 16 | d₂ | Length of the common perpendicular to X axes of |
| | | the connecting rods 3 and 2 |
| 17 | d₃ | Length of the common perpendicular to X axes of |
| | | the connecting rods 4 and 3 |
| 18 | M₀ | Reference system M where the dentition model is |
| | | located, and its origin is M₀ |

In an embodiment, the step of determining the coordinates of the dentition model in the bulb tube coordinate system and the rotation matrix between the world coordinate system and the bulb tube coordinate system according to the number of turns of rotation of each of the joint modules, the length of each of the joint modules, the length of each of the connecting rods, and the coordinates of the dentition model in the world coordinate system includes: determining an angle of rotation of each of the joint modules according to the number of turns of rotation of each of the joint modules; and determining the coordinates of the dentition model in the bulb tube coordinate system and the rotation matrix between the world coordinate system and the bulb tube coordinate system according to a DH coordinate system of the robotic arm and a transformation matrix. M0 denotes the origin (0,0) of the dentition model reference system. Because the dentition model is stationary in the world coordinate system, the coordinates of the dentition model in the world coordinate system are determined according to a distance between the dentition model and the origin (the stand column of the machine) of the base coordinate system.

Parameters of the DH coordinate system are described as follows: θᵢ denotes an angle between x axes of coordinate systems {Oᵢ₋₁} and {Oᵢ}; dᵢ denotes an offset of the coordinate system {Oᵢ} with respect to the coordinate system {Oᵢ₋₁} in the zᵢ₋₁ axis direction; αᵢ denotes an angle between a drive shaft and a transmission shaft of a connecting rod i; and aᵢ denotes the length of the connecting rod i in the mathematical sense. Values of these parameters are shown in table 2 below:

TABLE 2

| i | αᵢ₋₁ | aᵢ₋₁ | dᵢ | θᵢ₋₁ |
|---|---|---|---|---|
| 1 | 0 | 0 | 0 | θ₁ |
| 2 | −90° | a₁ | d₁ | θ₂ |
| 3 | 0 | a₂ | d₂ | θ₃ |
| 4 | −90° | 0 | d₃ | θ₄ |
| 5 | 90° | 0 | 0 | θ₅ |

According to the transformation matrix:

ᵢⁱ⁻¹T=rot_{z}θᵢtrans_{z}dᵢrotₓtransₓ(aᵢ)

a transformation matrix of each connecting rod coordinate system is obtained as follows:

₁⁰T=rot_{z}(θ₁)

₂¹T=rot_{z}θ₂trans_{z}d₁rotₓ(−90°)transₓ(a₁)

₃²T=rot_{z}θ₃trans_{z}d₂transₓ(a₂)

₄³T=rot_{z}θ₄trans_{z}d₃rotₓ(−90°)

₅⁴T=rot_{z}θ₅rotₓ(90°)

In a case where the coordinates of the origin M₀ of a dentition model coordinate system in the world coordinate system are known, coordinates ⁵ P_{M}_{₀} of M₀ in a coordinate system at the end of the teaching machine, namely, in the connecting rod coordinate system 5 are calculated, and a rotation matrix ₀^{W} T is as follows:

$${W}^{} {P}_{{M}_{0}} = {}_{5}^{W} {T}_{} {}_{}^{5} {P}_{{M}_{0}}^{} {}_{5}^{W} T = {}_{0}^{W} T {}_{1}^{0} T {}_{2}^{1} T {}_{3}^{2} T {}_{4}^{3} T {}_{5}^{4} T$$

$${}_{0}^{W} T = [ \begin{matrix}1 & 0 & 0 & 0 \\ 0 & 1 & 0 & 0 \\ 0 & 0 & 1 & {H}_{0} \\ 0 & 0 & 0 & 1\end{matrix} ]$$

where H₀ is the height (the vertical height of joint 1 to the ground) of the metal stand column of the teaching machine.

The dentition model is fixed on the desktop and does not move, and its coordinate system is a dentition model coordinate system {M}. The main body of the dental teaching platform is a five-axis robotic arm RRRRR, where the angle of rotation of each joint and the joint length are shown in FIG. 5. The final goal of calculation is to calculate the coordinates of the dentition model in the bulb tube coordinate system (namely, the coordinate system of the connecting rod 5), and a virtual periapical X-ray film of a corresponding tooth position can be obtained by rendering according to the coordinates and the related rotation matrix.

Step 4. Consistency Evaluation of Virtual Projection Images of Different Tooth Positions

In evaluation of the image quality of a periapical X-ray film, the following criteria are generally used internationally: 1) whether the image is stretched or shortened; 2) whether the image of the affected tooth is in the center of the periapical X-ray film; 3) whether the affected tooth overlaps the adjoining tooth; 4) whether the film is fully exposed; and the like. Likewise, quality evaluation is also required for virtual periapical X-ray films of different tooth positions. In order to simulate different situations that arise when students practice, such as image distortion caused by too large/small vertical angles, overlapping of tooth images, and the like, it is necessary to ensure that the virtual dental radiography machine can completely simulate the situations that arise in a real dental radiography machine. Before actually using this machine for periapical X-ray film training, it is required to determine whether such simulation can work exactly the same as a real dental X-ray machine. If there is an obvious deviation from the X-ray machine, the simulation will be ineffective for dental X-ray training or becomes worse due to repetition of wrong operations. In order to make comprehensive comparison between the novel dental radiography machine teaching platform and the original machine, it is required to ensure that there is no difference between the rendered image and the X-ray image under the same conditions. Therefore, the exact same environment needs to be set up for both machines, which means that the two machines must be set to have the same positions, the same angles, and the same postures, and especially can control the angles about the x, y and z axes simultaneously. On the premise of controlling the angles, a difference between the real X-ray film and the virtual periapical X-ray film is compared. In order to compare the difference, in the embodiment of the present application, parameters such as coefficients of variation in the tooth length and contour on the same tooth position are compared on the premise of controlling a projection angle.

The specific comparison method is as follows:

All of the following apparatuses and related experiments are in a standard radiation protection room. The operator must use a radiation protection device when performing periapical X-ray film projection.

The essential element of the steps is that the angle of each axis must be as precise as possible (the error must be less than 1 degree). After completion of these steps, the operator shall close the door of the radiation protection room and use the controller for both machines; and press the exposure buttons at the same time. Within one second, rendered images can be obtained from the computer. Afterwards, the operator puts the IP plate placed on the dental X-ray machine into an image reader. Therefore, comparison between the two images can be made. In order to make scientific comparison, the following two parameters are mainly considered: the root length and the overall contour offset degree. The total sample includes 12 different teeth, classified into the following groups: central incisors, posterior incisors, incisors, premolars, and molars. The intraoral radiography is done by a postgraduate in oral medicine. The used machines are the novel dental radiography machine teaching platform and a MAYO M machine having a working voltage of 70 kV and a working current of 7 mA. The exposure time for the molars and the premolars is 0.20 s, while the exposure time for anterior teeth is 0.14 s.

The tooth length is measured by using open-source image viewer Horos software, with a minimum unit of 0.1 mm. The distance from the buccal tip to the imaging root apex is measured using a length measurement tool in Horos. In the case of double-rooted and triple-rooted teeth, measurement is made to the apex of the longest root. After the measured reference tooth lengths are recorded, the tooth lengths measured by the two machines are compared in pairs, and a difference less than 1.0 mm in the measured root lengths is considered as a deviation between the two machines.

Contour differences between different tooth positions are compared by using matchShape( ) function in OpenCV; and it is considered that there is no obvious difference when the coefficient of variation in the contour is less than 0.2 (accuracy >80%).

Step 5. Integration of Functional Modules of the Virtual Dental Radiography Machine

The number-of-turns encoder for each joint will send the data to a micro network switch via a PLC. Finally, the mini workstation in the teaching platform calculates, in real time, the rotation matrix of the bulb tube coordinate system and the corresponding coordinates of the dentition model in the bulb tube coordinate system; and renders the corresponding virtual periapical X-ray film by means of software.

There are problems such as X-ray radiation, no direct image feedback, and low learning efficiency in the conventional teaching of angle-bisecting projection of the periapical X-ray film. By means of the robotic arm (having multiple connecting rods and joints) and the number-of-turns encoder, the present application can produce, by rendering in real time, a high-accuracy virtual periapical X-ray film with a precision up to one thousandth of a degree when the student operates the dental radiography machine teaching platform, thus greatly improving the learning efficiency of the student when learning the angle-bisecting projection technique and avoiding the harm from the X rays.

In the Lighthouse infrared positioning system in the prior art, infrared pulses are easily blocked by an obstacle, that is, the human body may affect the emission and reception of the infrared pulses, leading to a system failure. Moreover, the infrared positioning system requires determining the origin of the coordinate system and making correction. For non-professionals, the correction time is long and many corrections are required, making it difficult to operate. Different from the previous-generation Lighthouse system based on infrared pulse positioning, the novel periapical X-ray film teaching platform of the dental radiography machine in the present application adopts a new-generation mechanical positioning method, which has easy and direct operations and improved result accuracy.

Compared with the previous-generation Lighthouse system based on infrared pulse positioning, the virtual teaching system for a dental periapical X-ray film of the present application has parameters, such as the coefficients of variation in the tooth length and contour, closer to those of the real periapical X-ray film, as shown in table 3 below:

TABLE 3

| | Virtual teaching | |
|---|---|---|
| | system for a | Lighthouse infrared |
| | dental periapical | pulse-based |
| Comparative parameter | X-ray film | teaching platform |
| Coefficient of variation | <2% | 4~11% |
| in the tooth length | | |
| (maximum) | | |
| Coefficient of variation | <0.1 | <0.3 |
| in the contour | | |

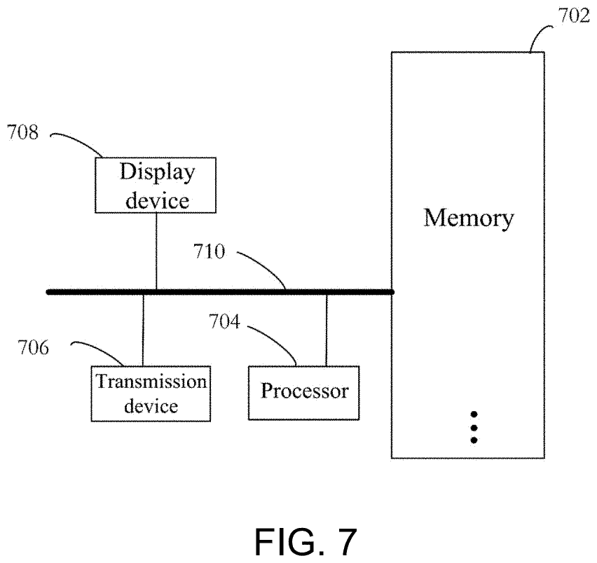

According to still another aspect of the embodiment of the present application, an electronic device for implementing the foregoing virtual periapical X-ray film acquisition method is further provided, where the electronic device can, but is not limited to, be applied in a server. As shown in FIG. 7, the electronic device includes a memory 702 and a processor 704, the memory 702 storing a computer program, where the processor 704 is configured to perform steps in any of the foregoing method embodiments by using the computer program.

Alternatively, in this embodiment, the electronic device may be disposed in at least one network device among multiple network devices of a computer network.

Alternatively, in this embodiment, the processor can be configured to perform the following steps by using the computer program:

S1. acquiring initial rendered images of a dentition model at different angles;
S2. taking an image of the dentition model at a preset position by using a bulb tube of a dental radiography machine, where the dental radiography machine includes a five-axis robotic arm, the five-axis robotic arm includes five joint modules and five connecting rods, and an end portion of the five-axis robotic arm is connected to the bulb tube;
S3. determining coordinates of the dentition model in a bulb tube coordinate system and a rotation matrix between a world coordinate system and the bulb tube coordinate system according to the number of turns of rotation of each of the joint modules, the length of each of the joint modules, the length of each of the connecting rods, and coordinates of the dentition model in the world coordinate system; and
S4. obtaining a virtual periapical X-ray film corresponding to the dentition model according to the coordinates of the dentition model in the bulb tube coordinate system, the rotation matrix between the world coordinate system and the bulb tube coordinate system, and a spatial posture and the Euler angle, and with reference to the initial rendered images of the dentition model.

Alternatively, persons of ordinary skill in the art can understand that the structure shown in FIG. 7 is merely schematic; and the electronic device may be a terminal device such as a smartphone (such as an Android phone, an iOS phone, etc.), a tablet, a palmtop, a Mobile Internet Device (MID), a PAD, or the like. FIG. 7 does not limit the structure of the foregoing electronic device. For example, the electronic device may further include more or fewer components (e.g., a network interface) than those shown in FIG. 7, or have a configuration different from that shown in FIG. 7.

The memory 702 can be used for storing software programs and modules, for example, program instructions/modules corresponding to the virtual periapical X-ray film acquisition method and device in the embodiments of the present application. The processor 704 executes various functional applications and data processing by running the software programs and modules stored in the memory 702, that is, implements the above-described virtual periapical X-ray film acquisition method. The memory 702 may include a high-speed random access memory; and may also include a non-volatile memory, such as one or more magnetic storage devices, a flash memory, or other non-volatile solid-state memories. In some examples, the memory 702 may further include memories located remotely from the processor 704, and these remote memories may be connected to the terminal via a network. Examples of the network include, but are not limited to, the Internet, intranet, local area network (LAN), mobile communication network, and a combination thereof. The memory 702 can specifically, but is not limited to, be used for storing the procedural steps of the virtual periapical X-ray film acquisition method.

Alternatively, the transmission device 706 is used for receiving or sending data via a network, where specific examples of the network include a wired network and a wireless network. In an example, the transmission device 706 includes a network adapter (Network Interface Controller, NIC), which can be connected to another network device and a router via a network cable and thus communicate with the Internet or the LAN. In an example, the transmission device 706 is a Radio Frequency (RF) module, which is used for communicating with the Internet in a wireless manner.

In addition, the electronic device further includes: a display device 708 used for displaying a virtual periapical X-ray film acquisition process; and a connection bus 710 used for connecting various module parts of the electronic device.

An embodiment of the present application further provides a computer readable storage medium, the storage medium storing a computer program, where the computer program is configured to execute the steps of any of the foregoing method embodiments during running.

Alternatively, in this embodiment, the storage medium can be configured to store a computer program for performing the following steps:

S1. acquiring initial rendered images of a dentition model at different angles;
S2. taking an image of the dentition model at a preset position by using a bulb tube of a dental radiography machine, where the dental radiography machine includes a five-axis robotic arm, the five-axis robotic arm includes five joint modules and five connecting rods, and an end portion of the five-axis robotic arm is connected to the bulb tube;
S3. determining coordinates of the dentition model in a bulb tube coordinate system and a rotation matrix between a world coordinate system and the bulb tube coordinate system according to the number of turns of rotation of each of the joint modules, the length of each of the joint modules, the length of each of the connecting rods, and coordinates of the dentition model in the world coordinate system; and
S4. obtaining a virtual periapical X-ray film corresponding to the dentition model according to the coordinates of the dentition model in the bulb tube coordinate system, the rotation matrix between the world coordinate system and the bulb tube coordinate system, and a spatial posture and the Euler angle, and with reference to the initial rendered images of the dentition model.

Alternatively, the storage medium is further configured to store a computer program for performing the steps included in the methods in the above embodiments, which is not repeated in this embodiment.

Alternatively, in this embodiment, persons of ordinary skill in the art can understand that all or some of the steps in the various methods of the foregoing embodiments can be done by instructing the hardware related to the terminal device through a program, and the program may be stored in a computer-readable storage medium. The storage medium may include a flash disk, a read-only memory (ROM), a random access device (RAM), a magnetic disk, or an optical disc.

The serial numbers in the foregoing embodiments of the present application are merely for the convenience of description, and do not imply the preference among the embodiments.

If the integrated unit in the foregoing embodiments is implemented in the form of software functional unit and is sold or used as an independent product, it can be stored in the above-mentioned computer-readable storage medium. Based on such understanding, the technical solution of the present application essentially, or the part contributing to the prior art, or all or part of the technical solution may be embodied in the form of a software product. The computer software product is stored in a storage medium, and includes instructions used for instructing one or more computer devices (which may be a personal computer, a server, or a network device, etc.) to perform all or some steps of the methods in various embodiments of the present application.

In the above embodiments of the present application, the description of each embodiment has its own emphasis. For content that is not detailed in one embodiment, reference may be made to related description in another embodiment.

In the several embodiments provided by the present application, it can be understood that, the disclosed client can be implemented in other ways. The device embodiments described above are merely illustrative. For example, the units are classified merely according to their logical functions, and may be classified in other manners during actual implementation. For example, multiple units or components may be combined or integrated into another system, or some features may be omitted or not implemented. In addition, the shown or discussed mutual coupling or direct coupling or communicative connection may be indirect coupling or communicative connection through some interfaces, units or modules in an electrical or other forms.

Units described as separate parts may or may not be physically separated. A part displayed as a unit may or may not be a physical unit, that is, the part may be located at one place or distributed on multiple network units. Some or all of the units may be selected according to an actual requirement to achieve the objective of the solution of the embodiments of the present application.

In addition, functional units in the embodiments of the present application may be integrated in one processing unit, or each unit may physically exist separately, or two or more units are integrated in one unit. The integrated units may be implemented in the form of hardware or a software functional unit.

The above merely describes preferred embodiments of the present application. It should be noted that, several improvements and modifications may be made by those of ordinary skill in the art without departing from the principle of the present application, and these improvements and modifications should also be construed as falling within the protection scope of the present application.

What is claimed is:

1. A virtual teaching system for dental periapical X-ray film, comprising a workstation and a dental radiography machine, wherein
the dental radiography machine comprises a mounting plate, a stand column, a head fixing device, a five-axis robotic arm, a bulb tube, and a seat; the stand column is disposed on a top side of the mounting plate, the head fixing device is fixed on an upper portion of the stand column, and the seat is fixed on a lower portion of the stand column; and the five-axis robotic arm comprises five joint modules and five connecting rods, one end of the five-axis robotic arm is connected to a top portion of the stand column and another end of the five-axis robotic arm is connected to the bulb tube; and
the workstation is communicatively connected to the dental radiography machine, so that the dental radiography machine positions standard teeth in a dentition model via the bulb tube, generates virtual projection image data of the standard teeth on a virtual plane, and sends the virtual projection image data to the workstation for rendering, to obtain a virtual periapical X-ray film corresponding to the dentition model.

2. The virtual teaching system for dental periapical X-ray film according to claim 1, wherein the five joint modules of the five-axis robotic arm comprises:
a first joint module, disposed between the stand column and a first connecting rod of the five connecting rods;
a second joint module, disposed between the first connecting rod and a second connecting rod of the five connecting rods;
a third joint module, disposed between the second connecting rod and a third connecting rod of the five connecting rods;
a fourth joint module, disposed between the third connecting rod and a fourth connecting rod of the five connecting rods; and
a fifth joint module, disposed between the fourth connecting rod and a fifth connecting rod of the five connecting rods, the fifth connecting rod being connected to the bulb tube,
wherein each of the joint modules is internally disposed with a multi-turn absolute encoder configured to acquire a number of turns of rotation of the corresponding joint module.

3. The virtual teaching system for dental periapical X-ray film according to claim 1, further comprising: a programmable logic controller (PLC) and a switch that are successively disposed between the dental radiography machine and the workstation, wherein a number of turns of rotation of each of the five joint modules is sent to the switch by means of the PLC and then sent to the workstation via the switch; and the workstation determines coordinates of the dentition model in a bulb tube coordinate system and a rotation matrix between a world coordinate system and the bulb tube coordinate system according to the number of the turns of the rotation of each of the five joint modules, a length of each of the five joint modules, a length of each of the five connecting rods, and coordinates of the dentition model in the world coordinate system, and then performs rendering to obtain the corresponding virtual periapical X-ray film.

4. The virtual teaching system for dental periapical X-ray film according to claim 1, wherein the dental radiography machine further comprises:
casters, mounted on a bottom side of the mounting plate.

5. A method for acquiring virtual periapical X-ray film, comprising:
acquiring initial rendered images of a dentition model at different angles;
taking an image of the dentition model at a preset position by using a bulb tube of a dental radiography machine, wherein the dental radiography machine comprises a five-axis robotic arm, the five-axis robotic arm comprises five joint modules and five connecting rods, and an end portion of the five-axis robotic arm is connected to the bulb tube;
determining coordinates of the dentition model in a bulb tube coordinate system and a rotation matrix between a world coordinate system and the bulb tube coordinate system according to a number of turns of rotation of each of the five joint modules, a length of each of the five joint modules, a length of each of the five connecting rods, and coordinates of the dentition model in the world coordinate system; and
obtaining a virtual periapical X-ray film corresponding to the dentition model according to the coordinates of the dentition model in the bulb tube coordinate system, the rotation matrix between the world coordinate system and the bulb tube coordinate system, and a spatial posture and an Euler angle of the bulb tube, and with reference to the initial rendered images of the dentition model.

6. The method for acquiring virtual periapical X-ray film according to claim 5, wherein determining the coordinates of the dentition model in the bulb tube coordinate system and the rotation matrix between the world coordinate system and the bulb tube coordinate system according to the number of the turns of the rotation of each of the five joint modules, the length of each of the five joint modules, the length of each of the five connecting rods, and the coordinates of the dentition model in the world coordinate system comprises:
determining an angle of the rotation of each of the five joint modules according to the number of the turns of the rotation of each of the five joint modules; and
determining the coordinates of the dentition model in the bulb tube coordinate system and the rotation matrix between the world coordinate system and the bulb tube coordinate system according to a DH coordinate system of the five-axis robotic arm and a transformation matrix.

7. The method for acquiring virtual periapical X-ray film according to claim 5, wherein after the virtual periapical X-ray film corresponding to the dentition model is obtained, the method further comprises:
determining whether there is a deviation between a real X-ray film and the virtual periapical X-ray film, wherein the real X-ray film is an X-ray film of the dentition model taken by an X-ray machine, and when the X-ray machine takes the real X-ray film, a placement position of the dentition model and an angle of the bulb tube are consistent with those when the dental radiography machine takes the virtual periapical X-ray film; and
if it is determined that there is the deviation, adjusting the placement position of the dentition model and the angle of the bulb tube according to the deviation between the real X-ray film and the virtual periapical X-ray film.

8. The method for acquiring virtual periapical X-ray film according to claim 5, wherein determining whether there is a deviation between the real X-ray film and the virtual periapical X-ray film comprises:
keeping an angle of the rotation of each of the five joint modules of the X-ray machine consistent with an angle of the rotation of each of the five joint modules of the dental radiography machine;
operating the X-ray machine and the dental radiography machine simultaneously, to obtain the real X-ray film and the virtual periapical X-ray film;
comparing the real X-ray film with the virtual periapical X-ray film in terms of a root length and an overall contour offset degree; and
if a difference in the root length is greater than 1 mm or a difference in the overall contour offset degree is greater than or equal to 0.2, determining that there is the deviation between the real X-ray film and the virtual periapical X-ray film.

9. A computer readable storage medium, storing a computer program, wherein the computer program is configured to perform the method for acquiring virtual periapical X-ray film according to claim 5 during running.

10. An electronic device, comprising a memory and a processor, the memory storing a computer program, wherein the processor is configured to perform the method for acquiring virtual periapical X-ray film according to claim 5 by running the computer program.
